(19) United States
(12) Patent Application Publication
(10) Pub. No.: US 2024/0180463 A1
(43) Pub. Date: Jun. 6, 2024

(54) Membrane Layer Containing a Therapeutic Agent for an Analyte Sensor

(71) Applicant: Celanese EVA Performance Polymers LLC, Irving, TX (US)

(72) Inventors: Jeffrey Haley, Norwood, OH (US); Harsh Patel, Union, KY (US); Philip Wilson, Cincinnati, OH (US)

(21) Appl. No.: 18/525,931

(22) Filed: Dec. 1, 2023

Related U.S. Application Data
(60) Provisional application No. 63/430,071, filed on Dec. 5, 2022.

(51) Int. Cl. A61B 5/1486; A61B 5/145

(52) U.S. Cl. CPC A61B 5/14865; A61B 5/14532

(57) ABSTRACT

An analyte sensor is disclosed. The analyte sensor includes an implantable sensor configured to measure a signal indicative of an analyte concentration in a user and at least one membrane layer. The membrane layer is disposed over at least a portion of the sensor. The membrane includes a membrane polymer matrix including an ethylene vinyl acetate copolymer and a therapeutic agent dispersed within the membrane polymer matrix. The membrane layer is permeable to an analyte. Devices and methods for monitoring analytes are also disclosed.

### RELATED APPLICATION

The present application is based upon and claims priority to U.S. Provisional Patent Application Ser. No. 63/430,071, having a filing date of Dec. 5, 2022, which is incorporated herein by reference.

### BACKGROUND

Continuous analyte sensors are utilized to provide health data regarding an analyte concentration in the body of a user. One example of a continuous analyte sensor includes a continuous glucose monitor. Continuous glucose monitors utilize a sensor that is inserted into the user such that the sensor can interact with interstitial fluid or blood of the user to determine the glucose concentration. Indeed, for users afflicted with diabetes mellitus, continuous monitoring of blood glucose levels is necessary. Many diabetics prick or cut their fingers in order to draw blood for analyte monitoring, which is painful and does not lend itself to continuous analyte monitoring.

As such, continuous glucose monitors have been developed that eliminate painful blood drawing and allow the user to more comfortable and continuously monitor glucose levels. Continuous glucose monitors utilize a sensor having a portion inserted into the user's tissue. However, during insertion a small wound site is formed around the inserted portion of the sensor. As such, surrounding bodily tissues can initiate an immune response, which can impair the function of the sensor. For instance, pro-inflammatory and pro-fibrotic factors can cause fibrotic tissue buildup on the external surface of the sensor that can prevent the analyte from being accurately detected by the sensor. When the sensor is no longer capable of accurate measurement, it must be changed by the user. Additionally, pathogens can infiltrate the wound at the insertion site causing pain or infection.

As such, there is a need for an improved analyte sensor that is capable of mitigating infection and immune response in a user.

### BRIEF SUMMARY

In accordance with one embodiment of the present disclosure, an analyte sensor is disclosed. The analyte sensor includes an implantable sensor configured to measure a signal indicative of an analyte concentration in a user. The analyte sensor includes at least one membrane layer located over the sensor. The membrane layer includes a membrane polymer matrix including an ethylene vinyl acetate copolymer and a therapeutic agent dispersed within the membrane polymer matrix. The membrane layer is permeable to an analyte. The ethylene vinyl acetate copolymer has a melt flow index of from about 1 to about 400 grams per 10 minutes as determined in accordance with ASTM D1238-20 at a temperature of 190° C. and a load of 2.16 kilograms.

In accordance with another embodiment of the present disclosure, devices for continuous in vivo measurement of an analyte concentration in a user are provided. The device includes an analyte sensor and one or more sensor circuitry operably connected to the analyte sensor configured to generate the analyte concentration. The analyte sensor includes an implantable sensor configured to measure a signal indicative of an analyte concentration in a user. The analyte sensor includes at least one membrane layer disposed on the sensor. The membrane layer includes a membrane polymer matrix including an ethylene vinyl acetate copolymer and a therapeutic agent dispersed within the membrane polymer matrix. The membrane layer is permeable to an analyte. The ethylene vinyl acetate copolymer has a melt flow index of from about 1 to about 400 grams per 10 minutes as determined in accordance with ASTM D1238-20 at a temperature of 190° C. and a load of 2.16 kilograms.

In accordance with another embodiment of the present disclosure, a method for forming a membrane layer on an analyte sensor is disclosed. The method includes disposing a composition comprising an ethylene vinyl acetate copolymer material, a therapeutic agent, optionally one or more hydrophilic compounds, and a solvent on a surface of the analyte sensor. The method includes evaporating the solvent to form a membrane layer on the sensor.

In accordance with another embodiment of the present disclosure, a method for continuously monitoring an analyte concentration in a user is disclosed.

Other features and aspects of the present disclosure are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of the analyte sensor of the present disclosure;

FIG. 2 is a cross-sectional view of the analyte sensor of FIG. 1;

FIG. 3 is a perspective view of one embodiment of the analyte sensor of the present disclosure;

FIG. 4 is a perspective view of one embodiment of the analyte sensor of the present disclosure;

FIG. 5 is a cross-sectional view of one embodiment of the analyte sensor of the present disclosure;

FIG. 6 is a perspective view of one embodiment of the analyte sensor of the present disclosure; and

FIG. 7 depicts a set of basic components of an analyte measuring device according to one embodiment of the present disclosure.

Repeat use of references characters in the present specification and drawing is intended to represent same or analogous features or elements of the disclosure.

### DETAILED DESCRIPTION

It is to be understood by one of ordinary skill in the art that the present discussion is a description of exemplary embodiments only, and is not intended as limiting the broader aspects of the present disclosure.

Generally speaking, the present disclosure is directed to an analyte sensor that is capable of continuously monitoring an analyte in a user (e.g., a patient) such as, a human, pet, farm animal, racehorse, etc. The analyte sensor includes a subcutaneous implantable sensor that is configured to measure a signal indicative of an analyte concentration in a user. Thus, the analyte sensor can be configured to provide for continuous or automatic monitoring of analytes in biological fluids (e.g., blood or interstitial fluid) of the user. Given that a portion of the sensor remains subcutaneously inserted in the user, the user may experience pain at the insertion site or fibrotic tissue can build up around the sensor impairing its performance. As such, at least one membrane layer is disposed on the sensor having a therapeutic agent dispersed therein. The therapeutic agent can be selected to reduce pain or inflammation at the insertion site, thus improving the performance or the lifetime of the sensor.

Various embodiments of the present disclosure will now be described in more detail.

### I. Membrane Layer(s)

a. Polymer Matrix

As indicated above, the analyte sensor includes one or more membrane layers (e.g., a first membrane layer) disposed on the analyte sensor. The membrane layer can be positioned adjacent to an outer surface of the analyte sensor. Additional membrane layers (e.g., a second membrane layer, a third membrane layer, etc.) may be layered on the analyte sensor as desired. The number of membrane layers may vary depending on the particular configuration of the sensor and/or the nature of the therapeutic agent. For example, in certain embodiments, the sensor may contain only one membrane layer, while in other embodiments, multiple membrane layers are present.

As indicated above, the polymer matrix contains at least a polymer that is generally hydrophobic in nature so that it can retain its structural integrity for a certain period of time when placed in an aqueous environment, such as the body of a mammal, and is stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the membrane polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-13 at a temperature of 190° C. and a load of 2.16 kilograms.

In certain embodiments, the membrane polymer matrix may contain a semi-crystalline olefin copolymer. The melting temperature of such an olefin copolymer may, for instance, range from about 40° ° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments about 20 wt. % to about 60 wt. %, and in some embodiments, from about 25 wt. % to about 50 wt. %. Conversely, the olefin monomeric content of the copolymer may likewise be within a range of from about 40 wt. % to about 90 wt. %, in some embodiments about 40 wt. % to about 80 wt. %, and in some embodiments, from about 50 wt. % to about 75 wt. %.

In one particular embodiment, for example, the membrane polymer matrix may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. In certain embodiments, the vinyl acetate content ranges from about 25 wt. % to about 32 wt. %. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm3), in some embodiments from about 0.910 to about 0.980 g/cm3, and in some embodiments, from about 0.940 to about 0.970 g/cm3, as determined in accordance with ASTM D1505-18. Particularly suitable examples of ethylene vinyl acetate copolymers that may be employed include those available from Celanese under the designation ATEVAR (e.g., ATEVAR 4030AC); Dow under the designation ELVAX® (e.g., ELVAX® 40 W); and Arkema under the designation EVATANE® (e.g., EVATANE 40-55). In embodiments, the ethylene vinyl acetate copolymer in the membrane polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and polymer matrix have a melting temperature and/or melt flow index within the range noted above. For example, the polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the polymer matrix.

In certain cases, ethylene vinyl acetate copolymer(s) constitute the entire polymer content of the membrane polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

b. Therapeutic Agents

As noted above, the membrane layer can include one or more therapeutic agents configured to control pathogens or reduce the immunological (e.g., inflammatory) response of the user. Various therapeutic agents can be incorporated into the membrane layer including anti-inflammatory agents, immunosuppressive agents, anti-microbial agents, anti-coagulation agents, or combinations thereof. In embodiments, the therapeutic agent includes one or more anti-inflammatory agents. Examples of anti-inflammatory agents suitable for use with the subject disclosure include, but are not limited to, peptide or protein anti-inflammatory agents (e.g., interleukin-1 receptor antagonist (IL-1RA/IL-1RA), non-steroidal anti-inflammatory drugs (NSAIDs) (e.g., salicylates, propionic acid derivatives, acetic acid derivatives, enolic acid (Oxicam) derivatives, fenamic acid derivatives, and the like). In certain embodiments, the anti-inflammatory agent is interleukin-1 receptor antagonist (IL-1RA/IL-1RA). In certain instances, the anti-inflammatory agent is a non-steroidal anti-inflammatory drug (NSAID), such as, but not limited to, a salicylate (e.g., aspirin (acetylsalicylic acid), diflunisal, salsalate, etc.), propionic acid derivatives (e.g., ibuprofen, naproxen, fenoprofen, ketoprofen, flurbiprofen, oxaprozin, etc.), acetic acid derivatives (e.g., indomethacin, sulindac, etodolac, ketorolac, nabumetone, etc.), enolic acid (Oxicam) derivatives (e.g., piroxicam, meloxicam, tenoxicam, lornoxicam, etc.), fenamic acid derivatives (e.g., mefenamic acid, meclofenamic acid, flufenamic acid, etc.), combinations thereof, and the like.

In certain embodiments, the anti-inflammatory agent can include steroidal agents. The term “steroidal agent” generally refers to a molecule capable of reducing and/or treating inflammation. Such steroidal agents may comprise one or more corticosteroids, such as glucocorticoids. Glucocorticoids are defined as a subgroup of corticosteroids. Glucocorticoids, sometimes also named glucocorticosteroids, are a class of steroid hormones that bind to the glucocorticoid receptor and are part of the feedback mechanism of the immune system that turns down immune activity, (e.g., inflammation). In medicine, they are used to treat diseases that are caused by an overactive immune system, such as allergies, asthma, autoimmune diseases, and sepsis. They also interfere with some of the abnormal mechanisms in cancer cells, so that they are also used to treat cancer. Upon binding, the glucocorticoid receptor, the activated glucocorticoid receptor complex up-regulates the expression of anti-inflammatory proteins in the nucleus by a process known as transactivation and represses the expression of pro-inflammatory proteins in the cytosol by attenuating actions on gene induction (via NF-κB, AP1, jun-jun-homodimers, etc.).

Suitable examples of glucocorticoids may comprise hydrocortisone, cortisone acetate, cortisone/cortisol, fluorocortolone, fluocinolone, flourometholone, prednisone, prednisolone, methylprednisolone, triamcinolone, dexamethasone, betamethasone, paramethasone, etc., as well as derivatives and combinations thereof. Dexamethasone and derivatives thereof are particularly suitable. Glucocorticoid polymorphs, isomers, hydrates, solvates, or derivatives thereof are all meant to be encompassed in the scope of the present disclosure and shall be understood to fall under the term “glucocorticoid”.

In embodiments, the therapeutic agent can include one or more immunosuppressants. Examples of immunosuppressants suitable for use with the subject disclosure include, but are not limited to, mammalian target of rapamycin (mTOR) inhibitors (e.g., everolimus, sirolimus, etc.), and the like. Other immunosuppressants suitable for use with the subject devices, methods and kits include, but are not limited to, drugs acting on immunophilins (e.g., ciclosporin, tacrolimus, sirolimus, everolimus, etc.), other immunosuppressive drugs (e.g., interferons, such as IFN-β; tumor necrosis factor-alpha (TNF-α) binding proteins, such as infliximab (Remicade), etanercept (Enbrel), or adalimumab (Humira); etc.), combinations thereof, and the like.

In other embodiments, the therapeutic agent can include an antimicrobial. As used herein, the terms “antimicrobial” or “antimicrobial agent”, refer to a substance or material that is detrimental (i.e., microbicidal) or microstatic (i.e., preventing or reducing colonization, expansion, and/or proliferation without necessarily being detrimental) to a microorganism, including bacteria, fungi, viruses, protozoans, and the like. Suitable antimicrobial agents include, but are not limited to, metals (e.g., silver), anti-biotics (e.g., cephalosporins, glycopeptide antibiotics, penicillins, quinolones, sulfonamides, tetracyclines), anti-virals (e.g., chemokine receptor antagonists, neuraminidase inhibitors, nucleoside reverse transcription inhibitors (NRTIs), protease inhibitors, purine nucleosides), anti-fungals (e.g., azole antifungals, echinocandins, polyenes), and combinations thereof.

In other embodiments, the therapeutic agent can include an anti-coagulation agent. As used herein, the anti-coagulation agent can include an agent that hinders the clotting of blood or interstitial fluids. As used herein, anti-coagulants can also include anti-platelets. Suitable anti-coagulants can include heparin, rivaroxaban, dabigatran, apixaban, edoxaban, warfarin, dipyridamole/ASA, fondaparinux, ticagrelor, prasugrel, clopidogrel, cliostazol, and combinations thereof.

c. Additives

Other additives may also be incorporated into the membrane layer(s) that are soluble and/or swellable in water. When employed, the weight ratio of the ethylene vinyl acetate copolymer(s) the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. In embodiments, the polyalkylene glycol has a molecular weight of from about 1,000 grams per mole to about 4,000 grams per mole. Specific examples of such polyalkylene glycols include, for instance, polyethylene glycols, polypropylene glycols polytetramethylene glycols, polyepichlorohydrins, etc.

Optionally, the membrane layer(s) can include a plurality of water-soluble particles distributed within a membrane polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

When employing multiple membrane layers, it is typically desired that each membrane layer contains a polymer matrix includes an ethylene vinyl acetate copolymer. Additionally, each of the membrane layers can include a plurality of water-soluble particles distributed within a membrane polymer matrix that includes an ethylene vinyl acetate copolymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain an ethylene vinyl acetate copolymer. The water-soluble particles and ethylene vinyl acetate copolymer(s) within one membrane layer may be the same or different than those employed in another membrane layer. In one embodiment, for instance, both the first and second membrane polymer matrices employ the same ethylene vinyl acetate copolymer(s) and the water-soluble particles within each layer have the same particle size and/or are formed from the same material. Likewise, the ethylene vinyl acetate copolymer(s) used in the membrane layer(s) may also be the same or different the hydrophobic polymer(s) employed in the core. In one embodiment, for instance, both the core and the membrane layer(s) employ the same ethylene vinyl acetate copolymer. In yet other embodiments, the membrane layer(s) may employ an ethylene vinyl acetate copolymer that has a lower melt flow index than a hydrophobic polymer employed in the core. Among other things, this can further help control the release of the therapeutic agent from the device. For example, the ratio of the melt flow index of a hydrophobic polymer employed in the core to the melt flow index of an ethylene vinyl acetate copolymer employed in the membrane layer(s) may be from about 1 to about 20, in some embodiments about 2 to about 15, and in some embodiments, from about 4 to about 12.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

### II. Formation

The membrane layer on the analyte sensor can be formed by any suitable process including solvent casting. Solvent casting refers to a process for forming a polymer layer or film by dipping a component into a solution composition containing the polymer and then drawing off solvent to leave a film (e.g., membrane layer) adhered to the component. Here, the analyte sensor can be dispersed in a solution composition containing the polymer, therapeutic agent, and other optional additives dispersed in a solvent. Once the solvent is drawn off, a membrane layer including the polymer, therapeutic agent, and other optional additives remains on the analyte sensor. While the component can be dipped into the solution composition, the disclosure is not so limited. Indeed, the solution composition can be sprayed, painted, rolled, or applied to the surface of the analyte sensor in any suitable manner. Application of the membrane layer can be controlled to ensure that the therapeutic agent is homogenously distributed throughout the membrane layer.

To form the membrane of the present disclosure, a solution composition is formed that includes the polymer material, therapeutic agent, and any desired additives, dispersed in a solvent. In such embodiments, the polymer material (e.g., ethylene vinyl acetate) can form about 10 wt. % to about 60 wt. % of the composition, such as from about 20 wt. % to about 50 wt. %, such as from about 30 wt. % to about 40 wt. % of the composition. The therapeutic agent can from about 1 wt. % to about 20 wt. % of the composition, such as from about 2 wt. % to about 19 wt. % of the composition, such as from about 3 wt. % to about 18 wt. % of the composition, such as from about 4 wt. % to about 17 wt. % of the composition, such as from about 5 wt. % to about 16 wt. % of the composition, such as from about 6 wt. % to about 15 wt. % of the composition, such as from about 7 wt. % to about 14 wt. %, such as from about 8 wt. % to about 13 wt. % of the composition, such as from about 9 wt. % to about 12 wt. % of the composition, such as about 10 wt. % of the composition. In embodiments, one or more additives (e.g., hydrophilic compounds) can also be added to the composition. In such embodiments, the additive can be present at from about 0 wt. % to about 20 wt. % of the composition, such as about 5 wt. % to about 15 wt. % of the composition, such as about 10 wt. % of the composition. The solvent is present in an amount of from about 50 wt. % to about 90 wt. % of the composition, such as about 60 wt. % to about 80 wt. % of the composition, such as about 70 wt. % of the composition.

The solvent can include any suitable solvent, including water, ethanol, methanol, acetone, tetrachloroethylene, toluene, methyl acetate, ethyl acetate, hexane, benzene, and combinations thereof. In embodiments, the solvent includes a polar organic solvent. Suitable polar organic solvents include acetone, acetonitrile, dimethylformamide (DMF), dimethylsulfoxide (DMSO), isopropanol, methanol, methylene chloride, and combinations thereof.

In embodiments, the solution composition can be repeatedly disposed on the analyte sensor in order to form additional layers or a thicker membrane layer on the analyte sensor. For instance, after solvent evaporation, additional solutions containing the polymer material, therapeutic agent and optional additives can be disposed on the surface of the analyte sensor. Similarly, when the solvent evaporates, an additional layer or thickness of membrane layer is formed on the analyte sensor. For instance, the solvent casting process can be continued until a desired membrane layer thickness is achieved. For instance, the membrane layer can have a thickness ranging from about 5 μm to about 50 μm, such as from about 10 μm to about 45 μm, 15 μm to about 40 μm, 20 μm to about 40 μm, 25 μm to about 30 μm.

In certain embodiments, the therapeutic agent can be homogenously distributed throughout the membrane layer. While in other embodiments, the therapeutic agent can be more concentrated at certain locations within the membrane layer. For instance, the therapeutic agent can have different concentration pockets distributed throughout the membrane layer. Still in other embodiments, the therapeutic agent is dispersed in a gradient manner in the membrane layer. For example, the concentration of the therapeutic agent can increase across the thickness of the membrane layer, while in other embodiments, the concentration of the therapeutic agent can decrease across the thickness of the membrane layer.

The desired concentration or dispersion of the therapeutic agent in the membrane layer can depend on several factors, including the analyte to be measured, location of placement of the device, and composition of the therapeutic agent. However, in embodiments, the membrane layer can include from about 5 wt. % to about 75 wt. % of the therapeutic agent, such as from about 10 wt. % to about 70 wt. %, such as from about 15 wt. % to about 65 wt. %, such as from about 20 wt. % to about 60 wt. %, such as from about 25 wt. % to about 55 wt. %, such as from about 30 wt. % to about 50 wt. %, such as from about 35 wt. % to about 45 wt. %. In other embodiments, the membrane layer can be loaded with from about 10 mg to about 100 mg of the therapeutic agent, such as from about 15 mg to about 95 mg, such as from about 20 mg to about 90 mg, such as from about 25 mg to about 85 mg, such as from about 30 mg to about 80 mg, such as from about 35 mg to about 75 mg, such as from about 40 mg to about 70 mg, such as from about 45 mg to about 65 mg, such as from about 50 mg to about 60 mg.

### III. Device Configurations

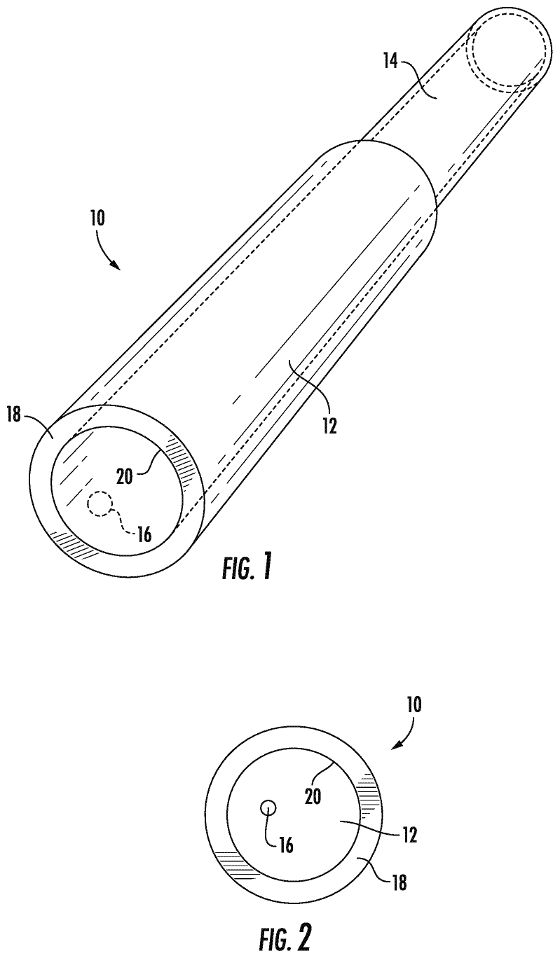

Referring now to FIGS. 1-2, for example, one embodiment of an analyte sensor 10 is shown. The analyte sensor includes a first portion 12 configured for insertion into the user. For example, the first portion 12 can be configured to be subcutaneously inserted into a user. The first portion 12 can extend from other portions of the analyte sensor 10 that are external to the user's skin. For instance, additional housings, adhesives, and other items may be disposed on the user's skin and the first portion 12 can extend from a housing disposed on the user's skin. A second portion 14 of the sensor 10 can remain external to the user's skin and can be coupled to a housing that is external to the user's skin (not shown). The first portion 12 is of a sufficient length in order to extend a desired depth into the user's tissue in order to access interstitial fluid or blood within the user's body. The first portion 12 can be provided in a variety of biological fluids in order to monitor the desired analyte. The first portion 12 can sense the desired analyte in dermal fluid, interstitial fluid, plasma, blood, lymph, synovial fluid, cerebrospinal fluid, saliva, bronchoalveolar lavage, amniotic fluid, or combinations thereof. The first portion 12 can be formed from any suitable materials, including polymeric materials and/or metal materials.

One or more sensing elements 16 can be disposed in the first portion 12. The sensing element 16 includes any element that is capable of monitoring the concentration of the desired analyte in biological fluid. For instance, sensing elements 16 are configured to provide data regarding measurement of an analyte, which can then be converted to determine the analyte concentration. Example sensing elements 16 are known and can include electrodes or additional sensing membrane layers. In embodiments, one or more electrodes can be disposed in or on the first portion 12 in order to monitor the analyte of interest. Such sensing elements are known by those of skill in the art.

Sensing elements 16 can further include any mechanism configured to facilitate measurement of an analytes. For instance, this can refer to known enzymatic or non-enzymatic mechanism by which an analyte can be quantified. In one example, the analyte sensor can be a glucose-measuring device that incorporates another membrane that contains glucose oxidase that catalyzes the conversion of oxygen and glucose to hydrogen peroxide and gluconate:

Glucose+O₂→Gluconate+H₂O₂

In the above reaction, for each glucose molecule consumed, there is a proportional change in the co-reactant O₂ and the product H₂O₂. Current change in either the co-reactant or the product can be monitored to determine glucose concentration. Additional membrane layers including sensing layers and sensors suitable for use in accordance with the present disclosure are described in U.S. Pat. No. 8,275,437, which is incorporated herein by reference.

A membrane layer 18 is disposed over at least a portion of the outer surface 20 of the first portion 12 of the analyte sensor 10. The membrane layer 18 can be disposed over at the entire outer surface 20 of the first portion 12. However, in other embodiments, the membrane layer 18 is only disposed over a certain percentage of the outer surface 20 of the first portion 12. In such embodiments, the membrane layer 18 can be configured to be disposed on at least 5% of the total surface area of the first portion 12, such as at least 10%, such as at least 20%, such as at least 30%, such as at least 40%, such as at least 50%, such as at least 60%, such as at least 70%, such as at least 80%, such as at least 90%, such as at least 100%.

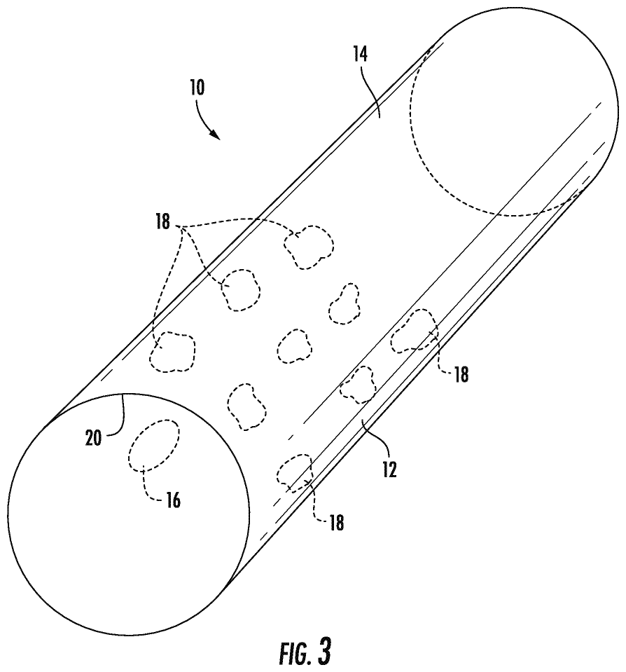

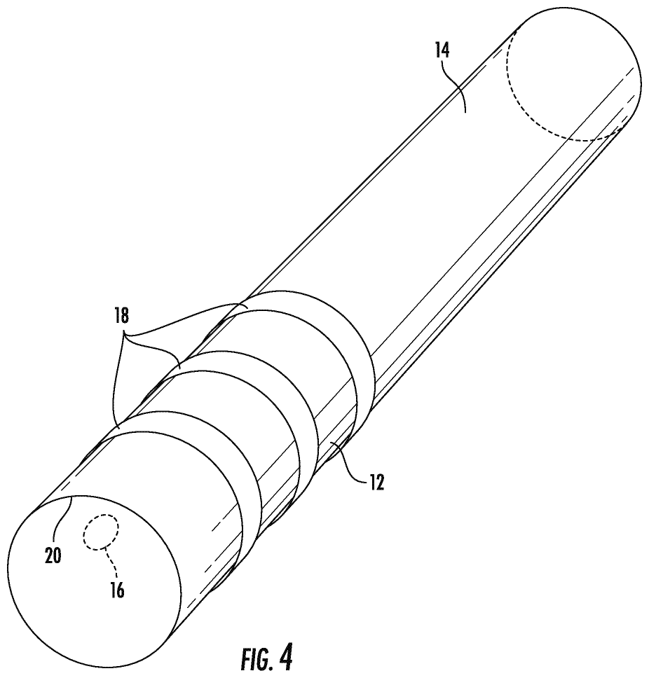

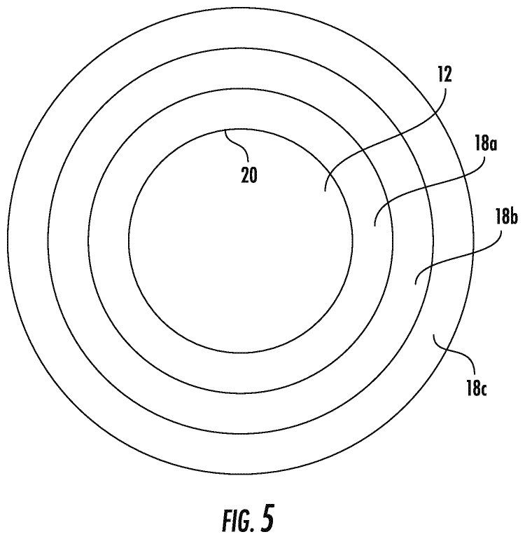

In certain embodiments, the membrane layer 18 can be disposed on the first portion 12 in a patterned manner. For instance, as shown in FIG. 3, discontinuous portions of the membrane layer 18 are disposed on the outer surface 20. Further, as shown in FIG. 4, the membrane layer 18 is disposed in discontinuous annular rings around the outer surface 20. Notably, while certain patterns are depicted herein the disclosure is not so limited and any suitable pattern can be used. For instance, depending on the selected therapeutic agent and sensor placement, various patterns of membrane layer 18 are contemplated.

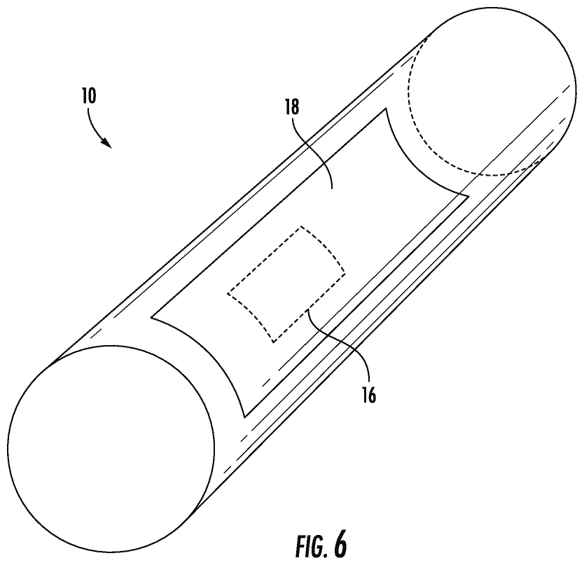

Now referring to FIG. 5, provided is an embodiment having multiple membrane layers 18a, 18b, 18c disposed on the outer surface 20 of the first portion 12. Notably, as disclosed herein, the membrane layers 18 include one or more therapeutic agents. In embodiments having multiple membrane layers 18, each membrane layer can include the same therapeutic agent or can include different amounts of the same therapeutic agent or can include different therapeutic agents. For instance, each membrane layer 18 can include a different concentration of the same therapeutic agent. In such an embodiment, membrane layer 18a can include the lowest concentration of therapeutic agent with membrane layer 18c having the highest concentration of therapeutic agent, thus creating a gradient of therapeutic agent from the outermost membrane layer to the innermost membrane layer. In other embodiments, membrane layer 18a can have the highest concentration of therapeutic agent and membrane layer 18c has the lowest amount of therapeutic agent. In certain embodiments, an additional membrane layer can be disposed on the membrane layer 18 (not shown). Such an additional membrane layer can be a rate limiting membrane layer configured to tune (e.g., limit) the release of the therapeutic agent from the membrane layer 18.

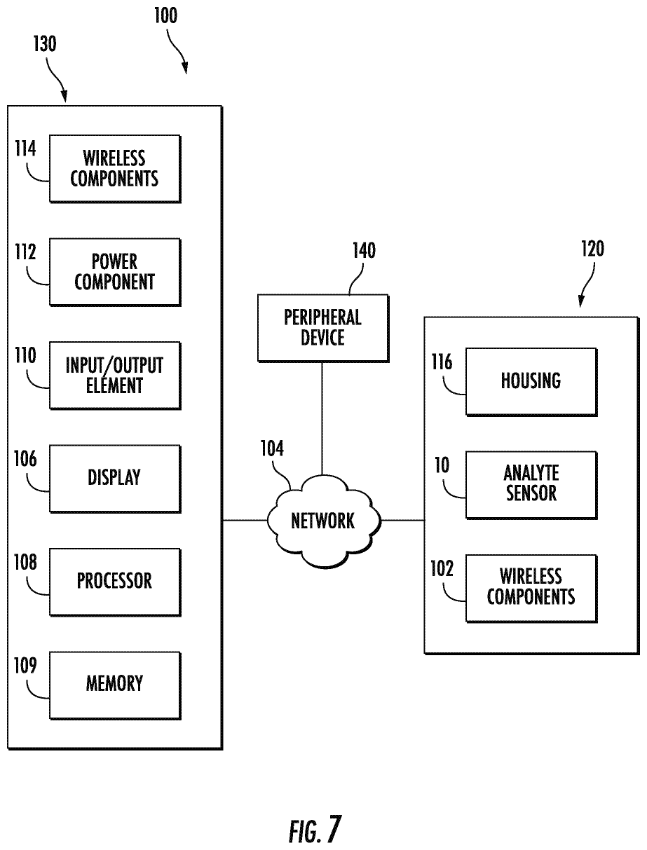

FIG. 6 illustrates a fully implantable analyte sensor 10 having a membrane layer 18 disposed thereon. In such embodiments, the analyte sensor 10 can include the membrane layer 18 on certain portions of the analyte sensor 10 or can include the membrane layer 18 over the entire surface of the analyte sensor 10 (not shown). For instance, the analyte sensor 10 can include a sensing element 16 and the membrane layer 18 is disposed over the sensing element 16.

Notably, the analyte sensor 10, including the membrane layer 18 of the present disclosure can be incorporated into a device configured to monitor analyte concentrations in a user. FIG. 7 illustrates a set of basic components 100 of one or more analyte monitoring devices of the present disclosure, in accordance with various embodiments of the present disclosure. Notably, the analyte monitoring device includes the analyte sensor 10 as described herein. The analyte sensor 10 is contained within a sensor assembly 120 and is operable connected to a sensor control unit 130. As would be apparent to one of skill in the art, the analyte monitoring device can include many types of memory 109, data storage or computer-readable media, such as a first data storage for program instructions for execution by the at least one processor 108, the same or separate storage can be used for images or data, a removable memory can be available for sharing information with other devices, and any number of communication approaches can be available for sharing with other devices. The sensor control unit 130 also includes one or more power components 112, such as a battery, including a rechargeable battery. The sensor control unit 130 may include at least one type of display 106, such as a touch screen, electronic ink (e-ink), organic light emitting diode (OLED) or liquid crystal display (LCD), although devices such as servers might convey information via other means, such as through a system of lights and data transmissions. The sensor control unit 130 typically will include one or more wireless components 114, such as a port, network interface card, or wireless transceiver that enables communication over at least one network 104. The sensor control unit 130 can also include at least one input device 110 able to receive input from a user. This input can include, for example, a push button, touch pad, touch screen, wheel, joystick, keyboard, mouse, trackball, keypad or any other such device or element whereby a user can input a command to the device. In certain embodiments, the input device 110 can include the display 106. While implementations are disclosed with reference to a processor 108, in some implementations an application specific integrated circuit can be utilized instead of or in addition to a processor. Indeed, the device can include any number of integrated circuits or circuitry necessary to perform processing tasks as needed.

The sensor assembly 120 is capable of detecting an analyte in the biological fluid of a user and transmitting data regarding the measurement of the analyte to the sensor control unit 130. The sensor assembly 120 can include the analyte sensor 10 as disclosed herein in addition to one or more wireless components 102 configured with a housing 116. For instance, as described hereinabove at least a portion of the analyte sensor 10 is disposed in contact with biological fluid of the host. In an example embodiment, a portion of the analyte sensor is subcutaneously disposed in a user and is in contact with the interstitial fluid of the user. Another portion of the analyte sensor 10 is coupled to a housing 116. The housing 116 can retain and include other elements such as additional processing circuitry or wireless components 102. The housing 116 can further include an adhesive portion for securing the sensor device 120 to the user. In the case of a continuous glucose monitor, the housing includes an adhesive portion that is configured to adhere the sensor assembly 120 to the skin of a user.

The wireless components 102 of the sensor assembly 120 can be connected via the network 104 to the wireless components 114 such that communication from the sensor assembly 120 to the processor 108 is facilitated. Protocols and components for communicating via such a network 104 are well known and will not be discussed herein in detail. Communication over the network 104 can be enabled via wired or wireless connections and combinations thereof. In some embodiments, the network includes the Internet, as the environment includes a Web server for receiving requests and serving content in response thereto, although for other networks, an alternative device serving a similar purpose could be used, as would be apparent to one of ordinary skill in the art.

The analyte sensor 10 can be implanted subcutaneously into the user and a portion of the analyte sensor 10 can be operable coupled to the sensor control unit 130. Via the analyte sensor 10, the sensor assembly 130 provides data that can then be correlated or converted to determine analyte levels in biological fluids, such as blood or interstitial fluid. In embodiments, the analyte sensor may be positioned in contact with interstitial fluid to detect the level of glucose, which detected glucose may be used to infer the glucose level in the user's bloodstream. Analyte sensors may be insertable into a vein, artery, or other portion of the body containing fluid.

When the analyte concentration is successfully determined, it can then be displayed, stored, transmitted, and/or otherwise processed to provide useful information. For example, the analyte concentration can be determined by the sensor control unit 130 and then can be displayed on the display 106. In other embodiment, the analyte concentration can be displayed on a peripheral device 140 via the network 104. Such a peripheral device 140 can include a user's smart phone, tablet, or computer. As such, the analyte sensor 10 can be included as part of a device for continuous in vivo measurement of an analyte concentration in a user. Notably, the device can further include sensor circuitry that is operably connected to the analyte sensor 10 and is configured to receive raw data from the analyte sensor 10 and then to convert that data to the analyte concentration. Such operations can be done by the processor 108.

### IV. Use of Device

As noted, the analyte sensor of the present disclosure can be inserted into a user and used to detect an analyte in bodily fluid. For instance, the analyte sensor is configured to detect any desired analyte, for example, glucose or other analytes (e.g., lactate, oxygen, pH, A1c, ketones, drug levels, toxins, etc.). Further, the term “analyte” as used herein is a broad term, and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the analyte sensor, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase 1; 17-alpha-hydroxyprogesterone; hypoxanthine phosphoribosyl transferase; immunoreactive trypsin; lactate; lead; lipoproteins ((a), B/A-1, 3); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, and 5-hydroxyindoleacetic acid (FHIAA).

The analyte sensor is useful in connection with methods of continuously monitoring an analyte in biological fluid of a user. In embodiments, the method can include measuring glucose in a user. The methods can also include measuring or monitoring another analyte (e.g., ketones, ketone bodies, HbA1c, and the like), including oxygen, carbon dioxide, proteins, drugs, or another moiety of interest, for example, or any combination thereof, found in bodily fluid, including subcutaneous fluid, dermal fluid (sweat, tears, and the like), interstitial fluid, or other bodily fluid of interest, for example, or any combination thereof. Such sensors that are implanted transcutaneously, subcutaenously, subdermally, etc. may be implanted by minimally invasive procedures. As used herein “trancutaneously” refers to a sensor wherein a least a portion of the sensor is disposed in or under one or more layers of the skin while at least another portion of the sensor remains external to the skin. Notably, in certain embodiments, the analyte sensor can be a fully implanted sensor. In such embodiments, no portion of the fully implanted sensor is external to the skin of the user. For instance, a fully implanted sensor can be fully inserted under the epidermis such that no portion of the sensor remains external to the skin. In such embodiments, the sensor can be disposed in the dermis or other subcutaneous tissue layers. Further, fully implantable sensors can be implanted into various tissues in the body (e.g., intra-organ, intra-tumoral, intra-vitreous) to monitor the desired analyte in the tissue.

Embodiments of the analyte sensors may be configured for monitoring the level of the analyte over a time period which may range from seconds, minutes, hours, days, weeks, to months, or longer. For instance, the analyte sensor can be configured to monitor the analyte for a time period of at least 1 days, such as at least 2 days, such as up to at least 7 days, such as up to at least 15 days, such as up to at least 30 days or more. In embodiments, the analyte sensor can be configured to monitor the level of the analyte for a time period of at least one month, such as at least 3 months, such as at least 6 months, such as at least 9 months, such as at least 12 months.

Upon insertion, the therapeutic agent may diffuse from the membrane layer into the surrounding tissues and can provide a local therapeutic effect in the tissues surrounding the analyte sensor. For instance, upon implantation of the analyte sensor the immune system may launch an immune response. In such a response, interleukins and cytokines are secreted by surrounding tissues. Additional pro-inflammatory and pro-fibrotic factors may also be released by tissues, especially those surrounding the analyte sensor. Release of immune response mediators by cells surrounding the tissue can interfere with the sensors ability to obtain an accurate measurement. These chemical mediators along with any fibrotic tissue build-up on the analyte sensor, decrease the sensitivity or the overall ability of the analyte sensor to obtain accurate measurements for conversion to an analyte concentration. As such, an accurate analyte concentration is not obtained. Release of therapeutic agents from the membrane layer, as disclosed herein, can decrease, or remediate the immunological response of localized cells, which can increase the performance (e.g., sensitivity) and operable life of the analyte sensor.

In embodiments, inclusion of the membrane layer including a therapeutic agent can increase the signal response over the life of the analyte sensor. For example, the analyte sensor can have an initial sensitivity. Over the operable lifetime of the analyte sensor, the analyte sensor, including the membrane layer as disclosed, can maintain a certain level of its initial sensitivity. For instance, the sensor can have a sensitivity that is 90% or more of the initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In other embodiments, the analyte sensor may maintain 95% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. Still in other embodiments, the analyte sensor maintains 97% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In still other embodiments, the sensor may maintain 99% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more.

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

What is claimed is:

1. An analyte sensor comprising:
an implantable sensor configured to measure a signal indicative of an analyte concentration in a user; and
at least one membrane layer disposed on at least a portion of the implantable sensor, wherein the membrane layer comprises a membrane polymer matrix including an ethylene vinyl acetate copolymer and one or more therapeutic agents dispersed within the membrane polymer matrix, wherein the at least one membrane layer is permeable to an analyte, wherein the ethylene vinyl acetate copolymer has a melt flow index of from about 1 to about 400 grams per 10 minutes as determined in accordance with ASTM D1238-20 at a temperature of 190° C. and a load of 2.16 kilograms.

2. The analyte sensor of claim 1, wherein the ethylene vinyl acetate copolymer has a melting temperature of from about 40° C. to about 120° C. as determined in accordance with ASTM D3418-15.

3. The analyte sensor of claim 1, wherein the ethylene vinyl acetate copolymer has a vinyl acetate content of from about 10 wt. % to about 60 wt. %.

4. The analyte sensor of claim 1, wherein the ethylene vinyl acetate copolymer in the membrane polymer matrix is from about 20 wt. % to about 99 wt. %.

5. The analyte sensor of claim 1, wherein the membrane polymer matrix includes a first ethylene vinyl acetate copolymer and a second ethylene vinyl acetate copolymer.

6. The analyte sensor of claim 1, wherein the one or more therapeutic agents includes a steroidal agent, an anti-inflammatory agent, an anti-coagulation agent, or combinations thereof.

7. The analyte sensor of claim 1, wherein the sensor has a generally circular cross-sectional shape.

8. The analyte sensor of claim 1, wherein one or more additional membrane layers are disposed between the at least one membrane layer and the sensor.

9. The analyte sensor of claim 8, wherein the one or more additional membrane layers comprise a polymer matrix composition that is different from the membrane polymer matrix.

10. The analyte sensor of claim 8, wherein the one or more additional membrane layers comprise a sensing layer comprising an analyte-responsive enzyme.

11. The analyte sensor of claim 8, wherein one or more additional membrane layers are disposed on an outer surface of the membrane layer.

12. The analyte sensor of claim 1, wherein the amount of therapeutic agent in the membrane polymer matrix is from about 5 wt. % to about 75 wt. %.

13. The analyte sensor of claim 1, wherein the membrane layer is loaded with about 10 mg to about 100 mg of the one or more therapeutic agents.

14. The analyte sensor of claim 1, wherein the membrane layer is capable of releasing the therapeutic agent for a time period of about 1 day to about 21 days or more.

15. The analyte sensor of claim 1, wherein the membrane polymer matrix comprises one or more hydrophilic compounds.

16. The analyte sensor of claim 15, wherein the one or more hydrophilic compounds are present in an amount of from about 1 wt. % to about 60 wt. %.

17. The analyte sensor of claim 15, wherein the one or more hydrophilic compounds comprises polyethylene glycol (PEG).

18. The analyte sensor of claim 1, wherein the analyte is glucose.

19. The analyte sensor of claim 1, wherein the analyte sensor is a transcutaneous analyte sensor.

20. A method for forming a membrane layer on an analyte sensor, the method comprising:
disposing a composition comprising a copolymer material, a therapeutic agent, optionally one or more hydrophilic compounds, and a solvent on a surface of the analyte sensor; and
evaporating the solvent to form the membrane layer.
